(19) United States
(12) Patent Application Publication
(10) Pub. No.: US 2024/0084016 A1
(43) Pub. Date: Mar. 14, 2024

(54) STABLE FORMULATION OF INTEGRIN ANTIBODY

(71) Applicant: DR. REDDY?S LABORATORIES LIMITED, Hyderabad (IN)

(72) Inventors: Murali JAYARAMAN, Kancheepuram, Tamil Nadu (IN); Swapnil Vasudeo PAKHALE, Jalgaon Maharashtra (IN); Bimlesh OJHA, Silchar, Assam (IN); Shrija GHOSH, Lucknow, Uttar Pradesh (IN)

(21) Appl. No.: 17/766,823

(22) PCT Filed: Oct. 10, 2020
(86) PCT No.: PCT/IN2020/050871

§ 371 Date: Apr. 6, 2022

(30) Foreign Application Priority Data
Oct. 11, 2019 (IN) 201941041231

(51) Int. Cl. C07K 16/28; A61K 9/19; A61K 47/10; A61K 47/18; A61K 47/26

(52) U.S. Cl. CPC C07K 16/2839; A61K 9/19; A61K 47/10; A61K 47/183; A61K 47/26

(57) ABSTRACT

The present invention discloses a stable pharmaceutical formulation of an α4β7 antibody, wherein the formulation contains buffer, PEG, salt, amino acid and surfactant and wherein the formulation is devoid of sugar and/or sugar alcohols. The disclosed antibody formulations are liquid formulations, and are also suitable to be formulated as a lyophilized powder.

### FIELD OF INVENTION

The present invention is related to stable formulations of an antibody molecule, wherein the antibody is stabilized with minimal excipients. The disclosed formulations are compatible with lyophilized as well as liquid form and also suitable for intravenous and/or subcutaneous route of administration.

### BACKGROUND

Over the past two decades, recombinant DNA technology has led to the commercialization of many proteins, particularly antibody therapeutics. The effectiveness of these therapeutic antibodies is majorly dependent on the stability, route of administration and their dosage forms and concentrations. This in turn, necessitates therapeutic antibodies to be formulated appropriately to retain the stability and activity of a therapeutic antibody.

Formulations for each route of administration and dosage forms may be unique and, therefore, have specific requirements. Solid dosage forms, such as lyophilized powders, are generally more stable than liquid (aqueous) formulations. However, reconstitution of the lyophilized formulation requires a significant vial overfill, care in handling and involves high production cost relative to a liquid formulation. While liquid formulations are advantageous in these and are usually preferred for injectable protein therapeutics (in terms of convenience for the end user and ease of preparation for the manufacturer), this form may not always be feasible given the susceptibility of proteins to denaturation, aggregation and oxidation under stresses such as temperature, pH changes, agitation etc.,. All of these stress factors could result in the loss of biological activity of a therapeutic protein/antibody. In particular, high concentration liquid formulations are susceptible to degradation and/or aggregation. Nevertheless, high concentration formulations may be desirable for subcutaneous or intravenous route of administration, as the frequency of administration and injection volume is reduced. On the other hand, specific treatment schedule and dosing might require a low concentration formulation and prefer intravenous route of administration for more predictable delivery and complete bioavailability of the therapeutic drug.

Hence, designing a formulation that is stable at high or low concentrations of the therapeutic protein/antibody, aiding in different route of administration (intravenous or subcutaneous) and which is suitable in lyophilized or liquid form, pose a significant developmental challenge. Further, every protein or antibody with its unique characteristics and properties of degradation, adds to the complexity in the development of a stable formulation and may demand a specific formulation.

A stable formulation of a therapeutic protein or antibody involves addition of a wide variety of stabilizers/excipients including amino acids, sugars, polyols, surfactants, salts, polymers, amines, anti-oxidants, chelators etc.,. Many of the FDA approved therapeutic proteins/antibodies contain more than one category of stabilizers.

A formulation combination with increased concentration of protein and/or stabilizers may increase the viscosity of the formulation, in turn increasing the injection time and pain at the site of injection and also pose difficulties during processing of the drug substance. Hence, it is necessary to develop an improved formulation, in lyophilized as well as liquid form which contains minimal number or concentration of excipients, yet stabilizing the drug at a wide range of its concentration.

### SUMMARY

The present invention discloses a stable pharmaceutical formulation of an antibody comprising, buffer, polyethylenglycol (PEG) and surfactant, wherein the said formulation is devoid of sugar(s).

In particular, the invention discloses a stable pharmaceutical formulation of an α4β7 antibody comprising buffer, PEG salt, free-amino acid and surfactant and wherein the said formulation is devoid of sugar(s). The antibody in the said formulation is stable and maintains at least 97% of monomeric content of the antibody in the formulation even after storage for four weeks at 40° C. or at 25° C.

The invention also discloses a method to control charge variants and aggregates of α4β7 antibody in an α4β7 antibody formulation by formulating the antibody in a buffer comprising PEG, salt, amino acid and surfactant components.

The combination of PEG, salt and free amino acid imparts colloidal stability to α4β7 antibody.

The disclosed combination of PEG, salt, amino acid and surfactant stabilizes the antibody at concentrations from about 60 mg/ml to about 200 mg/ml.

The combination of PEG, salt, free amino acid and surfactant inhibits the formation of high molecular weight aggregates of the antibody. Further, the particular combination inhibits the formation of charge variants (acidic/basic variants) content of the antibody.

In particular, the change in acidic variants content of the antibody for high concentration vedolizumab (at least 150 mg/ml) formulation is less than 5%, specifically less than 2%, even when the antibody samples are subjected to accelerated condition of 40° C. for 2 weeks.

The disclosed liquid formulations, are compatible with lyophilization process and no change in quality attributes (aggregates/acidic/basic variants) were seen even after lyophilization.

### DETAILED DESCRIPTION OF THE INVENTION

### Definitions

The term “about” refers to a range of values that are similar to the stated reference value and includes a range of values that fall within 20% or less, of the stated reference value.

The term “antibody” refers to a glycoprotein comprising at least two heavy (H) chains and two light (L) chains inter-connected by disulfide bonds, or an antigen-binding portion thereof. The “antibody” as used herein encompasses whole antibodies or any antigen binding fragment (i.e., “antigen-binding portion”) or fusion protein thereof.

The term “stable” formulation refers to the formulation wherein the antibody therein retains its physical stability and/or chemical stability and/or biological activity upon storage.

The term “initial” refers to the data at zero time point at the respective temperature. The data represented as ‘0’ W in the examples is the initial content of the antibody.

Stability studies provides evidence of the quality of an antibody under the influence of various environmental factors during the course of time. ICH's “Q IA: Stability Testing of New Drug Substances and Products,” states that data from accelerated stability studies can be used to evaluate the effect of short-term excursions higher or lower than label storage conditions that may occur during the shipping of the antibodies.

Various analytical methods are available for measuring the physical and chemical degradation of the antibody in the pharmaceutical formulations. An antibody “retains its physical stability” in a pharmaceutical formulation if it shows substantially no signs of aggregation, precipitation and/or denaturation upon visual examination of color and/or clarity, or as measured by UV light scattering or by size exclusion chromatography. An antibody is said to “retain its chemical stability” in a pharmaceutical formulation when its shows no or minimal formation of product variants which may include variants as a result of chemical modification of antibody of interest such as deamination, oxidation etc. Analytical methods such as ion exchange chromatography and hydrophobic ion chromatography may be used to investigate the chemical product variants.

The term ‘monomer’ as used herein describes antibodies consisting of two light chains and two heavy chains. The monomer content of an antibody composition is typically analyzed by size exclusion chromatography (SEC). As per the separation principle of SEC the large molecules or molecules with high molecular weight (HMW) elute first followed by smaller or lower weight molecules. In a typical SEC profile for an antibody composition, aggregates that may include dimers, multimers, etc., elute first, followed by monomer, and the clipped antibody variants or degradants may be eluted last. In some circumstances the aggregate peak or the degradant peaks may not elute as a baseline separated peaks but instead as a shoulder or abnormal broad peaks. In order to maintain the appropriate activity of an antibody, in particular of a therapeutic antibody, it is desirable to reduce the formation of aggregate or fragmentation of products and hence control the monomer content to a target value. Ability to inhibit the formation of aggregate and degradant content as measured at various time points during stability studies may indicate the suitability of the candidate formulation for antibody of interest. TSK-GEL G3000SWXL (7.8 mm×30 cm) column from TOSCH can be used on water HPLC to perform SEC.

The term ‘main peak’ as used herein refers to the peak that elutes in abundance (major peak) during a cation exchange chromatography. The peak that elutes earlier than the main peak, during a cation exchange chromatography, with a charge that is acidic relative to the main peak is termed acidic variant peak. The peak that elutes later than the main peak, during a cation exchange chromatography, with a charge that is relatively basic than the main peak is termed as basic variant peak. The main peak content can be determined by Ion exchange chromatography (IEC). There are two modes of IEC available viz., cation and anion exchange chromatography. Positively charged molecules bind to anion exchange resins while negatively charged molecules bind to cation exchange resins. In a typical cation exchange chromatographic profile of an antibody composition acidic variants elute first followed by the main peak and thereafter lastly the basic variants will be eluted. The acidic variants are a result of antibody modifications such as deamidation of asparagine residues. The basic variants are a result of incomplete removal of C-terminal lysine residue(s). In general, in an antibody a lysine residue is present at the C-terminal end of both heavy and light chain. An antibody molecule containing lysine at both heavy and light chain is referred to as K2 variant, the antibody molecule containing lysine residue at either one of heavy and light chain is referred to as K1 variant and antibody molecule having none is K0 molecule. Carboxypeptidase B (CP-B enzyme) enzyme acts on the C-terminal lysine residues present on K2 and K1 variants and thus converting them as K0 molecules. As per circumstances of the case, the IEC analysis can be carried out for samples digested with carboxypeptidase B (CP-B) enzyme. In a typical stability study it is expected that a stable formulation leads to reduction in formation of charge variants (acidic and basic variants), during the study, and hence minimize any reduction in main peak content.

Pharmaceutically acceptable excipients refer to the additives or carriers, which may contribute to stability of the antibody in formulation. The excipients may encompass stabilizers and tonicity modifiers. Examples of stabilizers and tonicity modifiers include, but not limited to, salts, surfactants, and derivatives and combination thereof.

The term sugar/s as used herein includes sugars and sugar alcohols/polyols. Sugars can be referred to monosaccharides, disaccharides, and polysaccharides. Examples of sugars include, but are not limited to, sucrose, trehalose, glucose, dextrose, raffinose and others.

Examples of sugar alcohols or polyols include, but are not limited to, mannitol, sorbitol, and others.

Surfactant refers to pharmaceutically acceptable excipients used to protect the protein formulations against various stress conditions, like agitation, shearing, exposure to high temperature etc. The suitable surfactants include but are not limited to polyoxyethylensorbitan fatty acid esters such as Tween 20™ or Tween 80™, polyoxyethylene-polyoxypropylene copolymer (e.g. Poloxamer, Pluronic), sodium dodecyl sulphate (SDS) and the like or combination thereof.

The term “free amino acid” as used herein refers to amino acid that is included in the formulation and is not a part of the buffer component. An amino acid may be present in its D- and/or L-form. The amino acid may be present as any suitable salt e.g. a hydrochloride salt, such as arginine-HCl.

Examples of salts include, but not limited to, sodium chloride, potassium chloride, magnesium chloride, sodium thiocyanate, ammonium thiocyanate, ammonium sulfate, ammonium chloride, calcium chloride, zinc chloride and/or sodium acetate.

The term “antioxidant” refers to an agent that inhibits the oxidation of other molecules and is not part of buffer component. Examples of antioxidants herein include citrate, lipoic acid, uric acid, glutathione, tocopherol, carotene, lycopene, cysteine, methionine, phosphonate compounds, e.g., etidronic acid, desferoxamine and malate.

Certain specific aspects and embodiments of the invention are more fully described by reference to the following examples. However, these examples should not be construed as limiting the scope of the invention in any manner.

### DETAILED DESCRIPTION OF THE EMBODIMENTS

Many of the approved antibody formulations contain sugar as a stabilizer. Specifically, non-reducing sugars such as sucrose, trehalose, mannitol, sorbitol are commonly and largely used as a stabilizer in many of the approved antibody formulations and these excipients are used in both lyophilized and liquid formulations. However, sugars, particularly sucrose, is known for degradation through hydrolysis during storage. Hydrolysis of sucrose molecule can be due to change in temperature or pH. Further, the hydrolyzed products of sucrose lead to glycation of the protein present in formulation. Despite this, sugar/sucrose plays a major role in stabilization and lyophilization of protein/antibody formulation and hence largely all the formulations include sugar/sucrose and has become difficult to exclude sugar/sucrose while at the same time develop a stable formulation.

However, the instant invention discloses an antibody formulation, specifically α4β7 antibody formulation, which is stabilized by PEG and without the presence of any sugar or sugar alcohol.

The present invention discloses a stable pharmaceutical formulation of an antibody comprising buffer, PEG, salt and surfactant, wherein the formulation is devoid of sugar.

In one embodiment, the invention discloses a stable pharmaceutical formulation of an α4β7 antibody comprising buffer, PEG, salt and surfactant, wherein the formulation is devoid of sugar and anti-oxidant.

In the above embodiment of the invention, the concentration of PEG present in the antibody formulation is at least 3%. Preferably, the concentration of PEG is 5%, 6%, 7%, 8%, 9% and 10%.

In an embodiment of the invention, the concentration of PEG in the antibody formulation is about 10%.

In any of the above said embodiments of the invention, α4β7 antibody formulation optionally contains free amino acid, preferably hydrophobic or basic amino acids.

In the above said embodiment, the hydrophobic amino acid is glycine and basic amino acid is arginine.

In all of the above mentioned embodiments of the invention, the concentration of the antibody in the formulation is about 50 mg/ml to about 200 mg/ml. Preferably the concentration of the antibody in the formulation is 60 mg/ml, or 80 mg/ml, or 100 mg/ml, or 120 mg/ml, or 150 mg/ml or 160 mg/ml, or 170 mg/ml or 175 mg/ml or 180 mg/ml.

In an embodiment, the invention discloses high concentration, α4β7 antibody formulation comprising about 150 mg/ml α4β7 antibody, buffer, PEG, salt, arginine and surfactant, and wherein the formulation is free of sugar and anti-oxidant.

In the above said embodiment, the concentration of PEG is at least 3% to about 10% and the weight by weight ratio of PEG to α4β7 antibody is 0.2-0.7:1 (w/w).

In an embodiment, the invention discloses high concentration α4β7 antibody formulation wherein the formulation comprising about 160 mg/ml vedolizumab, buffer, at least about 3% PEG, amino acid, salt and surfactant wherein the weight by weight ratio of PEG to antibody is 0.2: 1 (w/w).

In any of the above said embodiments of the invention, the α4β7 antibody formulation is stable and maintains at least 97% of monomeric content of the antibody, even after storage at 40° C. for four weeks and also aggregate content of the antibody is less than 2% even after storage at 40° C. for four weeks.

In any of the above said embodiments of the invention, the α4β7 antibody formulation is stable and contains less than 1.5% of low molecular weight (LMW) species or fragments in the formulation, even after storage at 40° C. for four weeks.

In any of the above said embodiments of the invention, the combination of PEG, amino acid, salt and surfactant present in the formulations inhibits/reduces the formation of charge variants even after storage at 40° C. for two weeks.

In an embodiment, the invention discloses a stable high concentration α4β7 antibody formulation, comprising about 150 mg/ml of α4β7 antibody, 20 mM histidine-phosphate buffer, 26 mg/ml arginine, 2. 92 mg/ml sodium chloride, 10% PEG and 0.6 mg/ml surfactant. The disclosed combination of excipients in the antibody formulation maintains at least 98% of monomeric content of the antibody composition even after storage at 40° C. for two weeks.

In an embodiment, the invention discloses a stable high concentration α4β7 antibody formulation comprising about 160 mg/ml α4β7 antibody, 50 mM histidine buffer, 26 mg/ml arginine, 2. 92 mg/ml sodium chloride, 10% PEG and 0.6 mg/ml surfactant. The disclosed combination of excipients in the antibody formulation maintains at least 98% of monomeric content of the antibody composition even after storage at 40° C. for two weeks.

In any of the above said embodiments of the invention, the combination of PEG, amino acid and salt present in the formulation imparts colloidal stability to the antibody formulation.

In any of the above said embodiments, the viscosity of formulations is less than 20 cP, specifically less than 10 cP.

In an embodiment the invention discloses, a method of controlling conversion of main peak content to charge variants and aggregate content of an α4β7 antibody, wherein the method comprises addition of a buffer composition comprising PEG, amino acid, salt and surfactant to the said antibody formulation.

In yet another embodiment, the invention discloses a method of controlling charge variants and aggregate content of α4β7 antibody in an α4β7 antibody formulation, the method comprising addition of a buffer composition comprising PEG, salt, arginine and surfactant to the antibody formulation, wherein the formulation controls formation of charge variants and aggregates of antibody and maintains at least 98% of monomeric content and at least 50% of the antibody as main peak content even when stored at 40° C. for two weeks or at 25° C. for four weeks.

In another embodiment, the invention discloses a method of reducing a change or increase in the charge variants and aggregate content of α4β7 antibody in an α4β7 antibody formulation, the method comprising addition of a buffer comprising PEG, salt, arginine and surfactant to the antibody formulation, wherein the change or increase in the aggregate content is less than 1% and charge variants content is less than 5%, when stored at 40° C. for two weeks or at 25° C. for four weeks, and when compared to the content at the initial storage conditions.

In the above mentioned embodiment, the change in acidic variants content is less than 5% even after storage at 40° C. for 2 weeks.

In the above said embodiment, the α4β7 antibody formulation is stable and maintains at least 98% of monomeric content of the antibody, even after storage at 40° C. for two weeks and also aggregate content of the antibody is less than 1.5% even after storage at 40° C. for two weeks.

In any of the above said embodiments, the α4β7 antibody formulated in combination of PEG, salt, arginine and surfactant is biologically active.

In any of the above mentioned embodiments, the salt present in α4β7 antibody formulation is sodium chloride.

In any of the above said embodiments, the buffer mentioned in the formulation includes organic buffer, inorganic buffer and/or combinations thereof.

In the above mentioned embodiment of the invention, the said organic buffer includes histidine buffer, succinate buffer or acetate buffer.

In yet another embodiment of the invention, the inorganic buffer mentioned in the formulation includes phosphate buffer.

In any of the above mentioned embodiments of the invention, the pH of α4β7 antibody formulation is from 5.0-7.0.

In one embodiment, the invention discloses a stable pharmaceutical formulation of α4β7 antibody comprising histidine-phosphate buffer, PEG, surfactant, salt and arginine, wherein the formulation does not contain sugar.

In the above said embodiment, the α4β7 antibody formulation is stable and maintains at least 97% of monomeric content of the antibody and controls the low molecular weight species to less than 1.2% in the formulation, even after storage at 40° C. for four weeks.

In an embodiment, the α4β7 antibody formulation comprising buffer, PEG, sodium chloride, surfactant and sugar, contains less than 15% of basic variants, and less than about 1.5% of low molecular weight species even after storage at 25° C. for four weeks.

In any of the above mentioned embodiments, the formulation of α4β7 antibody is a stable liquid (aqueous) formulation, which can be used for parenteral administration. Parenteral administration includes intravenous, subcutaneous, intra peritoneal, intramuscular administration or any other route of delivery generally considered to be falling under the scope of parenteral administration and as is well known to a skilled person.

In any of the above embodiments of the invention, the stable liquid/aqueous formulation is suitable and can be lyophilized as lyophilized powders. Further, the lyophilized formulation of α4β7 antibody can be reconstituted with appropriate diluent to achieve the liquid formulation suitable for administration.

In any of the above mentioned embodiments, the stable liquid α4β7 antibody are compatible with lyophilization process and the lyophilization process does not impact quality attributes of the antibody.

In the above mentioned embodiment, the lyophilized α4β7 antibody formulation is reconstituted using water for injection, wherein the reconstitution time is less than 5 minutes.

In any of the above mentioned embodiments, the α4β7 antibody includes vedolizumab.

Another aspect of the invention provides a vial, pre-filled syringe or autoinjector device, or any other suitable device comprising any of the subject formulations described herein. In certain embodiments, the aqueous formulation stored in the vial or pre-filled syringe or autoinjector device contains vedolizumab, PEG, buffer, amino acid, salt and surfactant.

In any of the above mentioned embodiments, α4β7 antibody formulations are visibly clear without any particles even when stored at 40° C. for two weeks or four weeks.

The disclosed formulations of the invention uses lesser amounts of excipients to stabilize the therapeutic antibody. Further, the formulations are devoid of sugar.

### EXAMPLES

An α4β7 antibody, vedolizumab, suitable for storage in the present pharmaceutical composition is produced by standard methods known in the art. For example, vedolizumab is prepared by recombinant expression of immunoglobulin light and heavy chain genes in a mammalian host cell such as Chinese Hamster Ovary cells. Further, the expressed vedolizumab is harvested and the crude harvest is subjected to standard downstream process steps that include purification, filtration and optionally dilution or concentration steps. For example, the crude harvest of vedolizumab may be purified using standard chromatography techniques such as affinity chromatography, ion-exchange chromatography and combinations thereof. The purified vedolizumab solution can additionally be subjected to one or more filtration steps, and the solution obtained is subjected to further formulation studies.

### Example 1 Vedolizumab Formulations without Sugars and Free Anti-Oxidants

Purified high concentration vedolizumab antibody approximately 100 mg/ml contains at least 8 mg/ml arginine and/or 75 mg/ml trehalose in histidine-phosphate buffer back ground was obtained from downstream chromatographic steps. Post which, depending on the requirement of excipients in the formulation PEG-400, NaCl, trehalose and polysorbate were added in the formulation to make final formulation. To maintain control, approximately 100 mg/ml of purified vedolizumab in histidine buffer back ground containing 26.3 mg/ml arginine, 100 mg/ml sucrose was obtained from downstream chromatographic steps. Polysorbate-80 was added to the final formulation. All formulations were diluted to make final formulation.

Details of all the five vedolizumab formulations are mentioned in Table 1. All vedolizumab formulations were subjected for accelerated stability studies at 40° C. for four weeks and at 25° C. for four weeks. Post which, the samples were analyzed for low molecular weight (LMW) species and monomer content using size exclusion chromatography (SEC) [results are given in Table 2 and 4] and also checked for main peak content, acidic, basic variants using ion-exchange chromatography [Table 3 and 5]. And also, these samples were checked for solubility and hydrodynamic diameter (Rh) using dynamic light scattering technique (DLS) [Table 6].

TABLE 1
Compositions of various vedolizumab formulations
without sugars and anti-oxidant

| Sample Name | Composition |
|---|---|
| Vmab-Control | Vedolizumab 60 mg/ml, 50 mM histidine |
| | monohydrochloride, sucrose 100 mg/ml, arginine 26.3 |
| | mg/ml, 0.6 mg/mL polysorbate 80, pH 6.2 |
| Vmab-1 | Vedolizumab 60 mg/ml, 20 mM histidine -phosphate |
| | buffer, 50 mM NaCl, 13 mg/ml arginine, 75 mg/ml |
| | trehalose, 0.6 mg/mL polysorbate 80, pH 6.2 |
| Vmab-2 | Vedolizumab 60 mg/ml, 20 mM histidine -phosphate |
| | buffer, 50 mM NaCl, 13 mg/ml arginine, 10% PEG-400, |
| | 0.6 mg/mL polysorbate 80, pH 6.2 |
| Vmab-3 | Vedolizumab 60 mg/ml, 40 mM histidine -phosphate |
| | buffer, 50 mM NaCl, 9.5 mg/ml arginine, 75 mg/ml |
| | trehalose, 0.6 mg/mL polysorbate 80 |
| Vmab-4 | Vedolizumab 60 mg/ml, 40 mM histidine -phosphate |
| | buffer, 50 mM NaCl, 9.5 mg/ml arginine, 10% PEG-400, |
| | 0.6 mg/mL polysorbate 80 |

TABLE 2
SEC data of vedolizumab (60 mg/ml) formulations prepared as per example 1

| | SEC data at 40° C. | | | | | | | | | | | |
|---|---|---|---|---|---|---|---|---|---|---|---|---|
| Sample | % of LMW at 40° C. | | | | % of monomer at 40 ° C. | | | | % of HMW | | | |
| Name | 0 W | 1 W | 2 W | 4 W | 0 W | 1 W | 2 W | 4 W | 0 W | 1 W | 2 W | 4 W |
| Vmab- | 0.0 | 0.3 | 0.7 | 1.2 | 99.6 | 99.0 | 98.1 | 97.0 | 0.4 | 0.7 | 1.1 | 1.8 |
| Control | | | | | | | | | | | | |
| Vmab-1 | 0.0 | 0.5 | 0.6 | 1.0 | 99.3 | 98.5 | 98.3 | 97.4 | 0.7 | 1.0 | 1.1 | 1.6 |
| Vmab-2 | 0.0 | 0.4 | 0.6 | 1.1 | 99.2 | 98.7 | 98.2 | 97.3 | 0.7 | 0.9 | 1.2 | 1.7 |
| Vmab-3 | 0.0 | 0.4 | 0.6 | 1.1 | 99.3 | 98.4 | 97.8 | 96.7 | 0.7 | 1.2 | 1.6 | 2.3 |
| Vmab-4 | 0.0 | 0.5 | 0.6 | 1.1 | 99.3 | 98.3 | 98.0 | 97.0 | 0.7 | 1.1 | 1.4 | 1.9 |

W—indicates weeks

TABLE 3
IEX data of vedolizumab (60 mg/ml) formulations
prepared as per example 1

IEX data at 40° C.

| | ‘0’ W | | | Change in % acidic |
|---|---|---|---|---|
| Sample | Acidic | Main | Basic | variants at 40° C. for |
| Name | variants | Peak | variants | four weeks |
| Vmab- | 21.6 | 67.9 | 10.5 | 20.6 |
| Control | | | | |
| Vmab-1 | 25.8 | 66.3 | 7.9 | 16.7 |
| Vmab-2 | 25.8 | 66.5 | 7.7 | 15.3 |
| Vmab-3 | 27.1 | 63.8 | 9.2 | 15.8 |
| Vmab-4 | 27.1 | 64.0 | 9.0 | 13.9 |

W—indicates weeks

TABLE 4
SEC data of vedolizumab (60 mg/ml) formulations
prepared as per example 1

| | SEC data at 25° C. | | | | | | | | |
|---|---|---|---|---|---|---|---|---|---|
| | % of LMW | | | % of Monomer | | | % of HMW | | |
| | at 25° C. | | | at 25° C. | | | at 25° C. | | |
| Sample | 0 | 4 | % | 0 | 4 | % | 0 | 4 | % |
| Name | W | W | change | W | W | change | W | W | change |
| Vmab- | 0.0 | 0.1 | 0.1 | 99.6 | 99.2 | 0.4 | 0.4 | 0.7 | 0.3 |
| Control | | | | | | | | | |
| Vmab-1 | 0.0 | 0.1 | 0.1 | 99.3 | 99.0 | 0.3 | 0.7 | 1.1 | 0.4 |
| Vmab-2 | 0.0 | 0.4 | 0.4 | 99.2 | 98.6 | 0.6 | 0.7 | 0.9 | 0.2 |
| Vmab-3 | 0.0 | 0.3 | 0.3 | 99.3 | 98.5 | 0.8 | 0.7 | 1.2 | 0.5 |
| Vmab-4 | 0.0 | 0.4 | 0.4 | 99.3 | 98.5 | 0.8 | 0.7 | 1.1 | 0.4 |

W—indicates weeks

TABLE 5
IEX data of vedolizumab (60 mg/ml) formulations prepared as per example 1

| | IEX data at 25° C. | | | | | | | | |
|---|---|---|---|---|---|---|---|---|---|
| Sample | % of Acidic variants at 25° C. | | | % of Main peak at 25° C. | | | % of Basic variants at 25° C. | | |
| Name | 0 W | 4 W | % change | 0 W | 4 W | % change | 0 W | 4 W | % change |
| Vmab- | 21.6 | 22.3 | 0.7 | 67.9 | 65.3 | 2.6 | 10.5 | 12.5 | 2.0 |
| Control | | | | | | | | | |
| Vmab-1 | 25.8 | 23.9 | −1.9 | 66.3 | 61.9 | 4.4 | 7.9 | 14.2 | 6.3 |
| Vmab-2 | 25.8 | 24.9 | −1.0 | 66.5 | 63.9 | 2.6 | 7.7 | 11.2 | 3.5 |
| Vmab-3 | 27.1 | 25.1 | −2.0 | 63.8 | 60.3 | 3.5 | 9.2 | 14.7 | 5.5 |
| Vmab-4 | 27.1 | 25.6 | −1.5 | 64.0 | 62.0 | 2.0 | 9.0 | 12.4 | 3.5 |

W—indicates weeks

TABLE 6
DLS data of vedolizumab (60 mg/ml) formulations
prepared as per example 1

| | DLS data at 40° C. | |
|---|---|---|
| | Diffusion co-efficient | Hydrodynamic radius |
| Sample | (Cm2/s) | (Rh) (nm) |

| Name | 0 W | 4W | 0 W | 4 W |
|---|---|---|---|---|
| Vmab- | 2.28E−07 | 2.46E−07 | 10.8 | 10.0 |
| Control | | | | |
| Vmab-1 | 2.16E−07 | 2.30E−07 | 11.4 | 10.7 |
| Vmab-2 | 2.21E−07 | 2.13E−07 | 11.2 | 11.6 |
| Vmab-3 | 2.07E−07 | 2.16E−07 | 11.9 | 11.4 |
| Vmab-4 | 2.15E−07 | 1.98E−07 | 11.4 | 12.4 |

W—indicates weeks

All the above formulations were also checked for change in pH. It was observed that there is no change in pH of the formulations even after storage for four weeks at 25° C. and at 40° C.

Further, these formulations were subjected for lyophilization and post which all the formulations were reconstituted and checked for reconstitution time, aggregate content using SEC and acidic species, basic species using IEX.

Reconstitution time for all five formulations were observed to be below 5 minutes. It has been observed that there is no change in aggregate content and acidic/basic species of all five formulation post reconstitution. The results clearly indicates that lyophilization process does not have any impact on quality attributes of the formulations.

### Example 2: High concentration vedolizumab formulations

Purified high concentration vedolizumab antibody approximately 100 mg/ml contains at least 8 mg/ml arginine and/or 75 mg/ml trehalose in histidine-phosphate/histidine buffer back ground was obtained from downstream chromatographic steps. Post which, depending on the requirement of excipients in the final formulation, buffer exchanged with a composition histidine/histidine-phosphate buffer with required amount of following excipients PEG-400, arginine and NaCl, Post buffer exchange, the formulations were concentrated upto 150-180 mg/ml. After, polysorbate 80 was spiked in all formulations. In the above step, one of the sample was exchanged with a composition containing PEG-400, arginine and salt without any buffer to know the stabilizing effect of this combination without buffer and concentrated up to 170 mg/ml.

To maintain control, approximately 100 mg/ml of purified vedolizumab in histidine buffer back ground containing 26.3 mg/ml arginine, 100 mg/ml sucrose was obtained from downstream chromatographic steps was buffer exchanged with a composition containing histidine buffer, arginine, and citrate. Post which, the antibody was concentrated up to 170 mg/ml. Polysorbate-80 was added to the final formulation. Approved high concentration vedolizumab formulation contains the above composition. Hence, maintained as control.

Details of all the four vedolizumab formulations are mentioned in Table 7. All vedolizumab formulations were subjected for accelerated stability studies at 40° C. for two weeks and at 25° C. for four weeks. Post which, the samples were analyzed for low molecular weight (LMW) species and monomer content using size exclusion chromatography (SEC) [results are given in Table 8] and also checked for main peak content, acidic, basic variants using ion-exchange chromatography [Table 9].

TABLE 7
Compositions of various high concentration vedolizumab
formulations without sugars and anti-oxidant

| Sample | |
|---|---|
| Name | Composition |
| Vmab- | Vedolizumab 175 mg/ml, 50 mM histidine |
| Control | monohydrochloride, arginine 26.3 mg/ml, 6.7 mg/ml |
| | sodium citrate, 0.5 mg/ml citric acid monohydro chloride, |
| | 0.6 mg/mL polysorbate 80, pH 6.2 |
| Vmab-6 | Vedolizumab 151 mg/ml, 20 mM histidine -phosphate |
| | buffer, 50 mM NaCl, 26 mg/ml arginine, 10% PEG-400, |
| | 0.6 mg/mL polysorbate 80, pH 6.2 |
| Vmab-7 | Vedolizumab 151 mg/ml, 50 mM histidine buffer, 50 mM |
| | NaCl, 26 mg/ml arginine, 10% PEG-400, 0.6 mg/mL |
| | polysorbate 80, pH 6.2 |

TABLE 8
SEC data of high concentration vedolizumab formulations prepared as per example 2

| | SEC data at 40° C. | | | | | | | | | | | |
|---|---|---|---|---|---|---|---|---|---|---|---|---|
| Sample | % of LMW at 40° C. | | | | % of monomer at 40° C. | | | | % of HMW | | | |
| Name | 0 W | 1 W | 2 W | Δ 2 W | 0 W | 1 W | 2 W | Δ 2 W | 0 W | 1 W | 2 W | Δ 2 W |
| Vmab- | 0.07 | 0.4 | 0.4 | 0.4 | 99.47 | 99.0 | 98.7 | −0.7 | 0.5 | 0.7 | 0.9 | 0.4 |
| Control | | | | | | | | | | | | |
| Vmab-6 | 0.05 | 0.4 | 0.5 | 0.5 | 99.33 | 98.5 | 98.0 | −1.3 | 0.6 | 1.1 | 1.5 | 0.9 |
| Vmab-7 | 0.08 | 0.4 | 0.6 | 0.5 | 99.36 | 98.7 | 98.2 | −1.2 | 0.6 | 0.9 | 1.2 | 0.6 |

W—indicates weeks,
Δ—indicates change

TABLE 9
IEX data of high concentration vedolizumab
formulations prepared as per example 2

| | IEX data at 40° C. | | | | | | | |
|---|---|---|---|---|---|---|---|---|
| Sample | % of Acidic variants at 40° C. | | | | % of main peak at 40° C. | | | |
| Name | 0 W | 1 W | 2 W | Δ 2 W | 0 W | 1 W | 2 W | Δ 2 W |
| Vmab- | 21.9 | 25.6 | 31.4 | 9.5 | 68.1 | 57.3 | 52.1 | −16.0 |
| Control | | | | | | | | |
| Vmab-6 | 22.3 | 21.2 | 24.2 | 1.9 | 66.4 | 52.8 | 47.0 | −19.4 |
| Vmab-7 | 22.7 | 23.5 | 27.1 | 4.4 | 67.7 | 56.1 | 51.2 | 16.5 |

W—indicates weeks,
Δ—indicates change

All the above formulations were also checked for change in pH. It was observed that there is no change in pH of the formulations even after storage for two weeks at at 40° C.

Further, all the samples were checked for visible particles. It was observed that, all the samples were clear, colorless without any visible particles 9^{th} October 2020 Signature:______
Dr. V R Srinivas
Dr. Reddy's Laboratories Ltd.

1. A stable pharmaceutical formulation of an a4b7 antibody comprising, buffer, PEG-400, arginine, salt and surfactant, wherein the formulation is free of sugar and anti-oxidant, and wherein the formulation stabilizes the antibody at concentrations from about 60 mg/ml to about 200 mg/ml.

2. The antibody formulation according to claim 1, wherein the buffer is histidine or histidine-phosphate buffer.

3. The antibody formulation according to claim 1, wherein the formulation maintains at least 97% of monomeric content of the antibody, even after storage at 25° C. for four weeks or at 40° C. for two weeks or at 40° C. for four weeks.

4. The antibody formulation according to claim 1, wherein the combination of PEG-400, arginine and salt reduces formation of high molecular weight species and charge variants.

5. A stable pharmaceutical formulation of a4b7 antibody comprising, about 160 mg/ml of a4b7 antibody, histidine buffer, 10% PEG, about 120 mM arginine, 50 mM sodium chloride and polysorbate 80.

6. The a4b7 antibody formulation according to claim 5, wherein the formulation maintains at least 98% of the antibody in its monomeric form even after storage at 40° C. for two weeks.

7. The a4b7 antibody formulation according to claim 5, wherein the formulation reduces formation of charge variants content and maintains at least 50% of the antibody in the formulation as main peak, even after storage at 40° C. for two weeks.

8. The antibody formulation according claim 1, wherein the antibody formulation is a liquid or lyophilized formulation.

9. A method of controlling aggregates and charge variants of an a4b7 antibody in an a4b7 antibody formulation, the method comprising addition of a buffer composition comprising PEG, salt, arginine and surfactant to the antibody formulation, wherein the formulation controls the increase in the aggregate content and acidic variant content of the antibody in the formulation, and maintains at least 98% of the antibody in monomeric content and at least 50% of the antibody as main peak content even when stored at 40° C. for two weeks or at 25° C. for four weeks.

10. The method according to claim 9, wherein the increase in the aggregate content of the antibody in the formulation is less than 1%, when compared to the aggregate content of the antibody at the initial storage conditions.

11. The method according to claim 9, wherein the increase in the charge variants content of the antibody in the formulation is less than 5%, when compared to the charge variants content of the antibody at the initial storage conditions.

12. The formulations according to claim 1, wherein the surfactant is polyosrbate 80.

13. The formulation according to claim 1, wherein the a4b7 antibody is vedolizumab.

14. The antibody formulation according claim 5, wherein the antibody formulation is a liquid or lyophilized formulation.

15. The formulations according to claim 5, wherein the surfactant is polyosrbate 80.

16. The formulations according to claim 9, wherein the surfactant is polyosrbate 80.

17. The formulation according to claim 5, wherein the a4b7 antibody is vedolizumab.

18. The formulation according to claim 9, wherein the a4b7 antibody is vedolizumab.
